(19) United States
(12) Patent Application Publication
(10) Pub. No.: US 2016/0214963 A1
(43) Pub. Date: Jul. 28, 2016

(54) INHIBITORS OF BRUTON'S TYROSINE KINASE

(71) Applicant: Beyond Oncology Pharmaceutical LLC, Pleasanton, CA (US)

(72) Inventor: Yi Chen, Pleasanton, CA (US)

(73) Assignee: Beyond Oncology Pharmaceutical LLC, Pleasanton, CA (US)

(21) Appl. No.: 15/088,336

(22) Filed: Apr. 1, 2016

Related U.S. Application Data
(63) Continuation-in-part of application No. PCT/US2014/056050, filed on Sep. 17, 2014.
(60) Provisional application No. 61/887,449, filed on Oct. 7, 2013.
(60) Provisional application No. 61/886,657, filed on Oct. 4, 2013.

(51) Int. Cl. C07D 403/10; C07D 401/10

(52) U.S. Cl. CPC C07D 403/10; C07D 401/10

(57) ABSTRACT

The disclosure includes compounds of Formula (I)

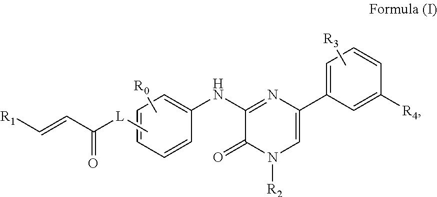

wherein R₀, R₁, R₂, R₃, R₄, R₅, and L are defined herein. Also disclosed is a method for treating a neoplastic disease, autoimmune disease, and inflammatory disorder with these compounds.

### RELATED APPLICATIONS

This application is a continuation of International Application No. PCT/US2014/056050, filed on Sep. 17, 2014; which claims the benefit of the filing date of U.S. Provisional Application 61/886,657, filed on Oct. 4, 2013; and U.S. Provisional Application 61/887,449, filed on Oct. 7, 2013. The entire contents of each of the above applications are incorporated herein by reference in their entirety.

### BACKGROUND

Bruton tyrosine kinase (Btk) is a Tec family non-receptor protein kinase, expressed in most hematopoietic cells such as B cells, mast cells, and macrophages but not in T cells, natural killer cells, and plasma cells (Smith, C. I. et al., Journal of Immunology (1994), 152 (2):557-565). Btk is a crucial part of the BCR and FcR signaling pathway, and the targeted inhibition of Btk is a novel approach for treating many different human diseases such as B-cell malignancies, autoimmune disease, and inflammatory disorders (Uckun, Fatih M. et al., Anti-Cancer Agents in Medicinal Chemistry (2007); Shinohara et al., Cell (2008) 132:794-806; Pan, Zhengying, Drug News & Perspectives (2008), 21 (7):357-362; Gilfillan et al., Immunological Reviews (2009) 288:149-169; Davis et al., Nature, (2010) 463:88-94).

Two successful approaches have been used to design highly selective BTK inhibitors. The first approach is to design irreversible inhibitors that covalently bind to an amino acid residue found in BTK but uncommon in the kinome (Pan, Zhengying et al., ChemMedChem, (2007) 2(1):58-61; Potashman, M. H., Duggan, M. E., J. Med. Chem., (2009) 52:1231-1246; Singh, J. et al., Current Opinion in Chemical Biology (2010), 14(4):475-480; Singh, J. Nat. Rev. Drug Discovery (2011) 10:307-317). Analysis of 491 kinase sequences for positional similarity within the ATP binding pocket revealed that Cysteine residue C481 of BTK is the amino acid most unique to BTK. The irreversible BTK inhibitors, such as PCI-32765 and AVL-292 (structures shown below), typically has an acrylamide moiety that bind irreversibly to C481. Interestingly, several well-known irreversible EGFR inhibitors such as HKI-272 and BIBW-2992 (structures shown below) also contains the acrylamide moiety which will irreversibly bind to cysteine residue occurs at similar position of EGFR. These findings clearly demonstrate that interaction with C481 is a valid approach for the design of kinome selective BTK inhibitors.

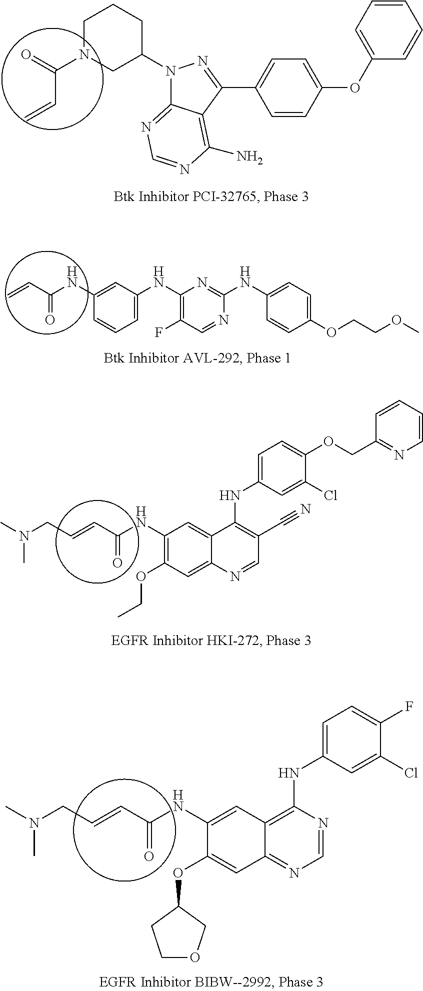

PCI-32765 is the most advanced irreversible Btk inhibitor approved for mantle cell lymphoma (MCL) and chronic lymphocytic leukemia (CLL), and in clinical development for a variety of B-cell malignancies including small lymphocytic lymphoma (SLL), and diffuse large B-cell lymphoma (DLBCL) and multiple myeloma (MM). PCI-32765 is a very potent (IC₅₀=0.5 nM) irreversible inhibitor of BTK in which acrylamide moiety irreversibly bind to residue C481 of Btk, thus achieving a >100-fold selectivity window in vitro over kinases with amino acids other than cysteine or serine at this position, and it achieves maximum potency against BTK and two other kinases that have the C481 residue. In addition, the preclinical studies of PCI-32765 in mice have shown that reduces the level of circulating autoantibodies and can reverse the course of arthritis. PCI-32765 also inhibited auto-antibody production and the development of kidney disease in a mouse model of systemic lupus erythematosus (SLE).

The second approach is to design selective BTK inhibitors that fill a “Specificity Pocket,” also referred as the “H3 pocket,” about 15 Å away from the hinge region. This Btk pocket presumably responsible for the high selectivity is formed to a large extent by residues S543, V546, and Y551 of the fully ordered activation loop. Sequence analysis of 491 human kinases shows that the V546/Y551 pair occurs in only three other kinases, BMX, ITK, and TXK. The triple S543/V546/Y551 is completely unique to Btk. CGI-1746 and RN-486 are two examples of Btk inhibitors tightly filling into the specificity pocket.

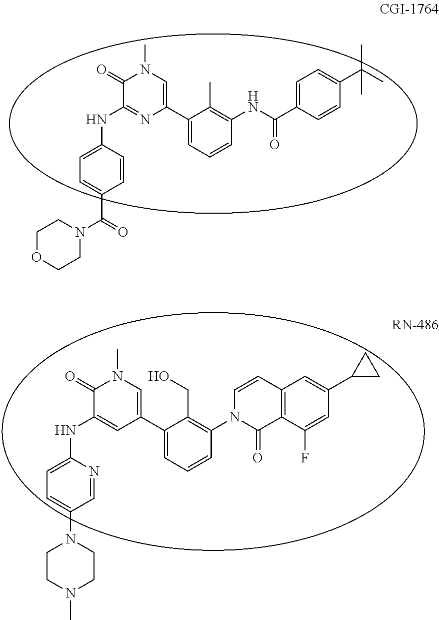 [SMILES: Cc1c(NC(=O)c2ccc(C(C)(C)C)cc2)cccc1-c1cn(C)c(=O)c(Nc2ccc(C(=O)N3CCOCC3)cc2)n1 ; CN1CCN(c2ccc(Nc3cc(-c4cccc(-n5ccc6cc(C7CC7)cc(F)c6c5=O)c4CO)cn(C)c3=O)nc2)CC1]

CGI-1746 is a potent (IC₅₀=1.9 nM) inhibitor of BTK tightly filing the specific pocket with 1000-fold selectivity over Tec and Src family kinases. The high kinome selectivity of the BTK inhibitor CGI-1746 stems from its tert-butylphenyl that occupies the specificity pocket with great complementarity. Competition binding of CGI-1746 in the Ambit panel of 385 kinases at 1 μM reveals only five kinases with more than 50% displacement of control ligand (BTK 100%, CASK 69%, MINK 77%, NEK11 59%, PDGFRB 59%). CGI-1746 binds MINK1, the second most tightly bound kinase, with a K_{d} of 40 μM vs 1.5 nM for BTK. A biochemical screen of 47 purified kinases at Kₘ for ATP showed that BMX, with IC₅₀=1.87 μM, was the next most potently inhibited kinase after BTK, with a nearly 1000-fold reduction in potency relative to BTK (Di Paolo, Nature Chemical Biology (2011) 7(1):41-50).

RN-486 is another very potent (IC₅₀=0.3 nM) inhibitor of BTK tightly filing the specific pocket, exhibiting a high degree of selectivity (>139-fold) over a large panel of 396 kinases (Yan Lou et al., J. Med. Chem., DOI: 10.1021/jm500305p). The high kinome selectivity of the RN-468 stems from its cycloalkyl moiety that occupies the specificity pocket with great complementarity. The data showed that RN-486 blocks both BCR and FcR signaling, inhibiting IgM-stimulated CD69 expression in B cells in human whole blood, IgG-FcγR mediated TNFα release in monocytes, and IgE-Fce cross-linking induced histamine release in mast cells with IC₅₀ values of 17, 4, and 29.2 nM, respectively. RN-486 dose-dependently inhibits disease progression in both mCIA and mCAIA models, as well as demonstrates an additive effect of inhibiting inflammation and bone erosion in adjuvant-induced arthritis. When RN-486 was administered orally in a preventive mode at doses of 3, 30, and 100 mg/kg in a mouse CIA model, it completely inhibited ex vivo anti-IgD stimulated CD69 expression at 3 and 6 h postdose at all doses and by approximately 50-80% 24 h postdose. RN-486 showed complete inhibition of arthritis as measured by clinical scores at 100 mg/kg, similar to dexamethasone. When RN-486 was studied in a therapeutic mode in mCIA and mCAIA models, animals treated with RN-486 did not show progression of disease at 30 mg/kg. In addition, RN-486 demonstrated a greater effect (91%, 30 mg/kg) than cyproheptadine, a nonselective antihistamine agent (69% maximum inhibition) in blocking the hypersensitive immune responses in a rat passive cutaneous anaphylaxis (rPCA) model. In addition, observed additive effects between RN-486 and low dose methotrexate in a rat AIA model demonstrated the potential of combination therapies for BTK inhibitors. (Xu Daigen, et al., The Journal of Pharmacology and Experimental Therapeutics (2012) 341(1):90-103)

Although BTK inhibitors such as PCI-32765, AVL-292, CGI-1746, and RN-486 have made a significant contribution to the art, there is a continuing search in this field of art for improved pharmaceuticals.

### SUMMARY OF THE INVENTION

The present invention relates to a class of selective Btk inhibitors which are rationally designed to not only irreversibly bind to the unique Btk residue C481 but also tightly fill the Btk specificity pocket formed by residues S543, V546, and Y551. More specifically, the Btk inhibitors in present invention contain an acrylamide moiety which will irreversible bind to residue C481 and a pharmcophore that will tightly fill into the specificity pocket. Such Btk inhibitors may possess highly favourable potency and selectivity. Thus, the compounds of the present invention may be useful in treating the patients with diseases such as B-cell malignancies, autoimmune disease, or inflammatory disorders.

In one aspect, this invention relates to a compound of Formula (I) or an N-oxide thereof, or a pharmaceutically acceptable salt, solvate, polymorph or tautomer of said compound of formula (I) or N-oxide thereof:

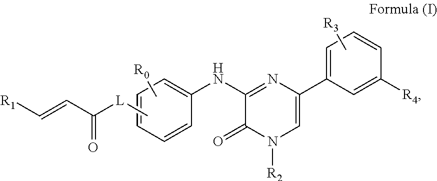

wherein

R₀ and R₁, independently, is H, alkyl, alkenyl, alkynyl, cycloalkyl, cycloalkenyl, heterocycloalkyl, heterocycloalkenyl, aryl, heteroaryl, halo, nitro, oxo, cyano, ORₐ, SRₐ, alkyl-Rₐ, alkyl-NR_{b}R_{c}, NR_{b}R_{c}, C(O)Rₐ, S(O)Rₐ, SO₂Rₐ, P(O)R_{b}R_{c}, C(O)N(R_{b})R_{c}, N(R_{b})C(O)R_{c}, C(O)ORₐ, OC(O)Rₐ, SO₂N(R_{b})R_{c}, or N(R_{b})SO₂R_{c}, in which each of Rₐ, R_{b}, and R_{c}, independently, is H, alkyl, alkenyl, alkynyl, cycloalkyl, cycloalkenyl, heterocycloalkyl, heterocycloalkenyl, aryl, heteroaryl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, dialkylamino, or alkylamino;

L is —N(R_{d})(CH₂)ₘ in which R_{d} is H, alkyl, alkenyl, alkynyl, cycloalkyl, heterocycloalkyl, and each of m is 0, 1, 2, 3, or 4;

R₂ is H or alkyl;

R₃ is H, halo, alkyl, or hydroxyalkyl;

R₄ is

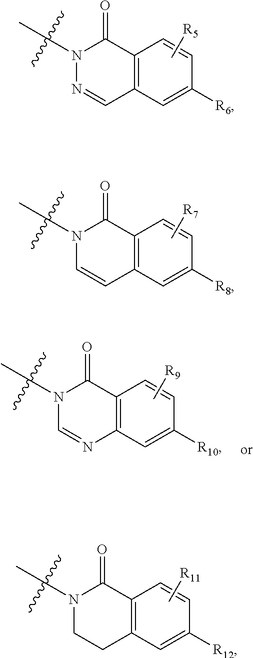

in which each of R₅, R₆, R₇, R₈, R₉, R₁₀, R₁₁, and R₁₂ independently, is H, alkyl, alkenyl, alkynyl, cycloalkyl, heterocycloalkyl, halo, or alkoxy; and

In certain embodiments, the compound is represented by

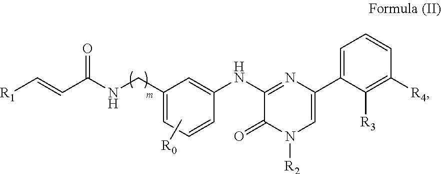

In certain embodiments, R₄ is

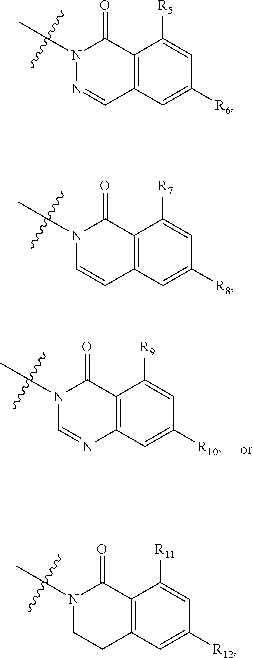

In preferred embodiments, L is —NH(CH₂)ₘ; R₁ is H, alkyl, alkyl-Rₐ, alkyl-NR_{b}R_{c}; R₂ is H, methyl, or ethyl; R₃ is H, methyl, or hydroxymethyl; and R₅, R₆, R₇, R₈, R₉, R₁₀, R₁₁, and R₁₂, independently, is H, alkyl, alkenyl, alkynyl, cycloalkyl, heterocycloalkyl, or halo.

In a more preferred embodiment, R₁ is H or alkyl-NR_{b}R_{c}; R₂ is methyl; R₃ is hydroxymethyl; R₆, R₈, R₁₀, and R₁₂, independently, is H, alkyl, or cycloalkyl; and R₅, R₇, R₉, and R₁₁ independently, is H, alkyl, or halo.

In a more preferred embodiment, R₅, R₇, R₉, and R₁₁ independently, is H or F.

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, particularly the B-cell malignancy including but not limited to B-cell lymphoma, lymphoma (including Hodgkin's and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc.) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:

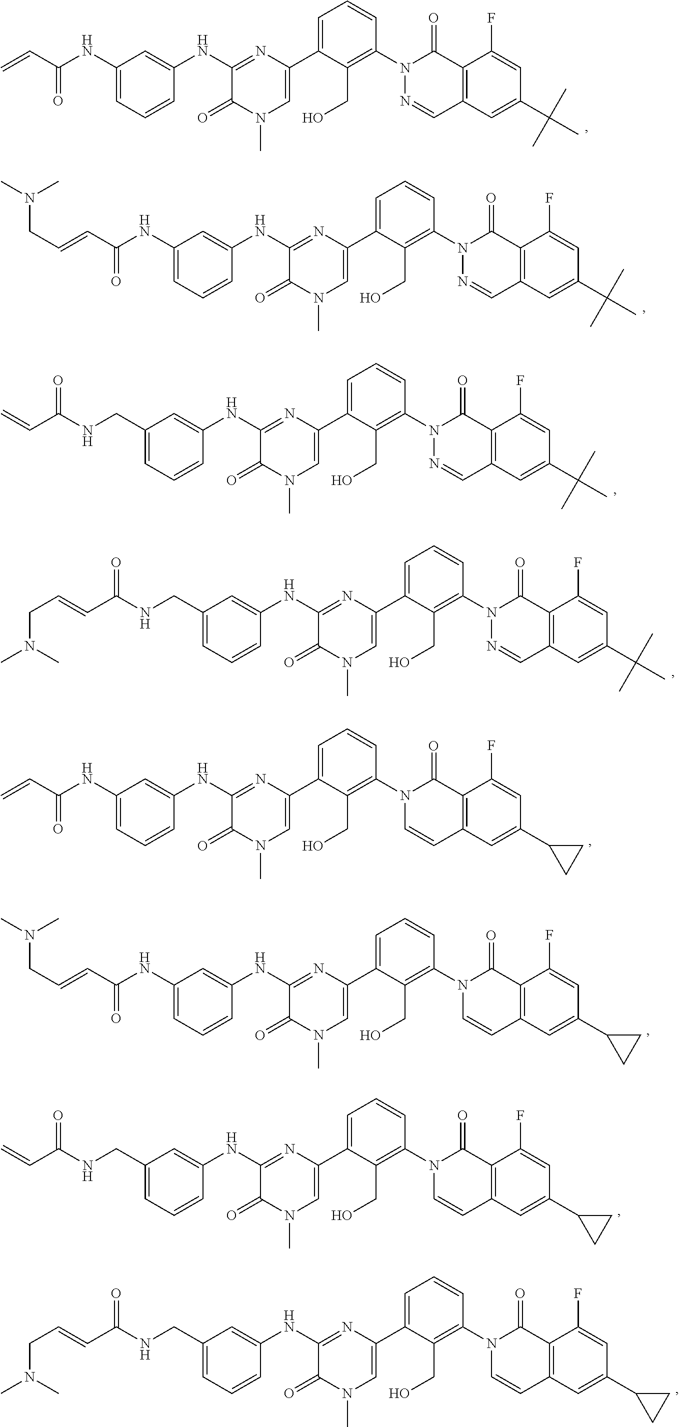

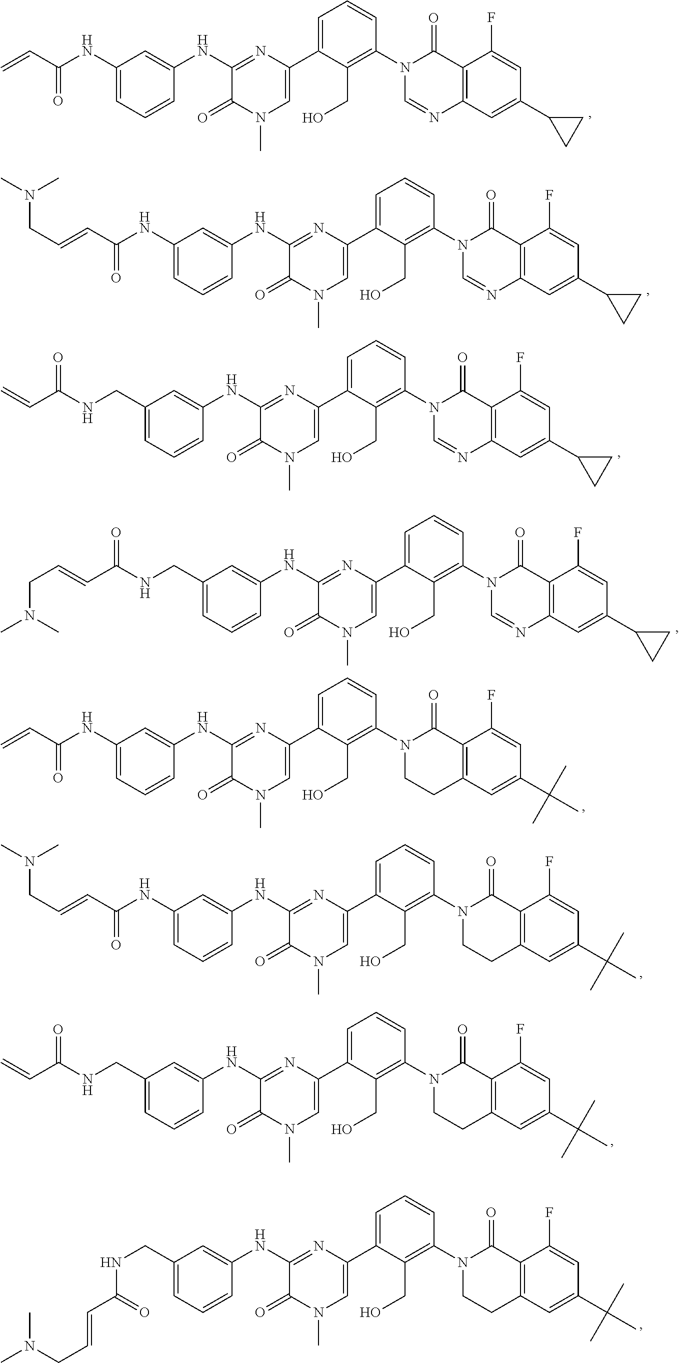

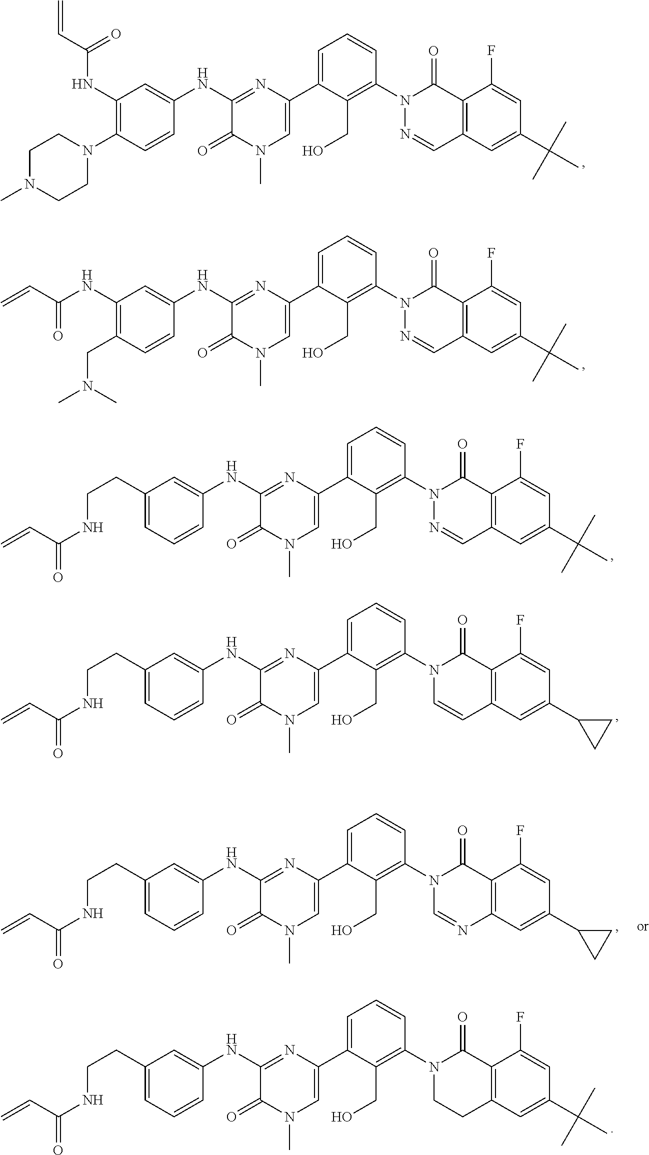

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention (Nature Reviews of Drug Discovery, 2008, Volume 7, p. 255). It is noted that in many instances, the prodrugs themselves also fall within the scope of the range of compounds according to the present invention. The prodrugs of the compounds of the present invention can be prepared by standard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985.
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs (Mol. Cancer Therapy, 2004 March; 3(3):233-244). Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS 15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g., acetyl), aroyl (e.g., benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a nonaromatic 5-8 membered monocyclic, 8-12 membered bicyclic, or 11-14 membered tricyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, or 11-14 membered tricyclic ring system having one or more heteroatoms (such as O, N, S, P, or Se) and one or more double bonds.

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl,” as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors (Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDR/VEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6 KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES 1, ZAK/MLTK, ZAP70, ZIPK/DAPK3, KINASE, MUTANTS, ABL1(E255K), ABL1(F317I), ABL1(G250E), ABL1(H396P), ABL1(M351T), ABL1(Q252H), ABL1(T315I), ABL1(Y253F), ALK (C1156Y), ALK(L1196M), ALK (F1174L), ALK (R1275Q), BRAF(V599E), BTK(E41K), CHK2(I157T), c-Kit(A829P), c-KIT(D816H), c-KIT(D816V), c-Kit(D820E), c-Kit(N822K), C-Kit (T670I), c-Kit(V559D), c-Kit(V559D/V654A), c-Kit(V559D/T670I), C-Kit (V560G), c-KIT(V654A), C-MET(D1228H), C-MET(D1228N), C-MET(F1200I), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (I2020T), LRRK2 (R1441C), p38a(T106M), PDGFRa(D842V), PDGFRa(T674I), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g., HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g., Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc.).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate (e.g., brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc.).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARP inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alpha2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as etanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, Chagas disease, chronic obstructive pulmonary disease, chronic Idiopathic thrombocytopenic purpura (ITP), Churg-Strauss syndrome, Crohn's disease, dermatomyositis, diabetes mellitus type 1, endometriosis, Goodpasture's syndrome (and associated glomerulonephritis and pulmonary hemorrhage), Graves' disease, Guillain-Barré syndrome, Hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and Wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

A typical approach to synthesize the Formula (A) compounds

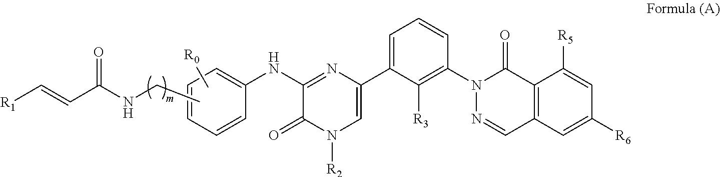

is described in Scheme A. R₀, R₁, R₂, R₃, R₅, R₆, and m in general Scheme A are the same as those described in the Summary section above.

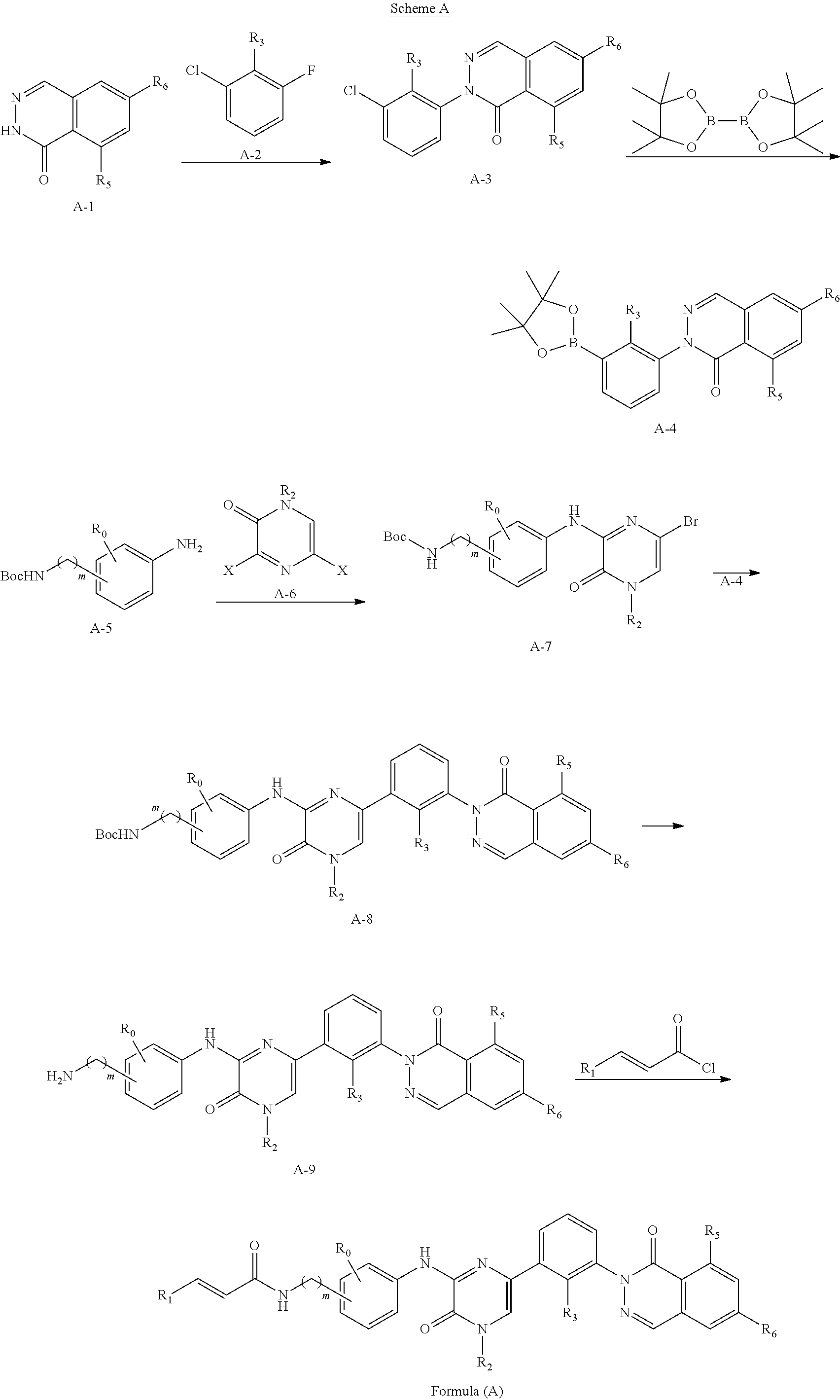

In Scheme A, the starting material A-1 can react with an appropriate 2-subsituted 1,3-halo-benzene A-2 to form the intermediate A-3, which can react with 4,4,4′,4′,5,5,5′,5′-octamethyl-2,2′-bi(1,3,2-dioxaborolane) to afford the intermediate A-4. Meanwhile, the appropriate amine intermediate (A-5) can react with 1-subsituted 3,5-dihalo-pyrazin-2(1H)-one (A-6), to yield the intermediate (A-7), which can couple with A-4 to form the intermediate A-8. Finally, the de-protection of intermediate (A-8) will affords the amine intermediate A-9, which can react appropriate acryloyl chloride to afford Formula (A).

Many kinds of starting material A-1 are commercially available. Alternatively, A-1 can be synthesized by various standard organic reactions, e.g., by the scheme of

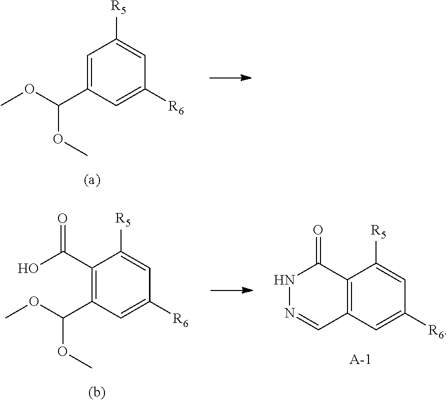

The formylation of an aryl bromide using a combination of a Grignard reagent and an alkyl lithium at a non-cryogenic temperature to yield the compound (a), after that the ortholithization of compound (a) followed by a carboxylation reaction to form the compound (b); finally the cyclizing the (b) with hydrazine will yield A-1.

Similarly, a typical approach to synthesize the Formula (A1) compounds

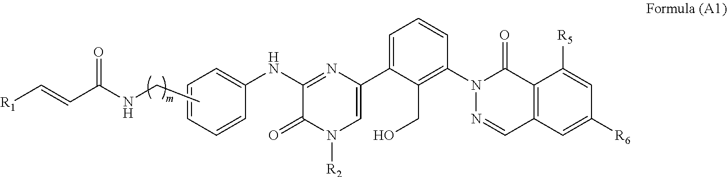

(R₂ is methyl, R₃ is hydroxylmethyl) is described in Scheme A-1. R₁, R₅, R₆, m in general Scheme B are the same as those described in the Summary section above.

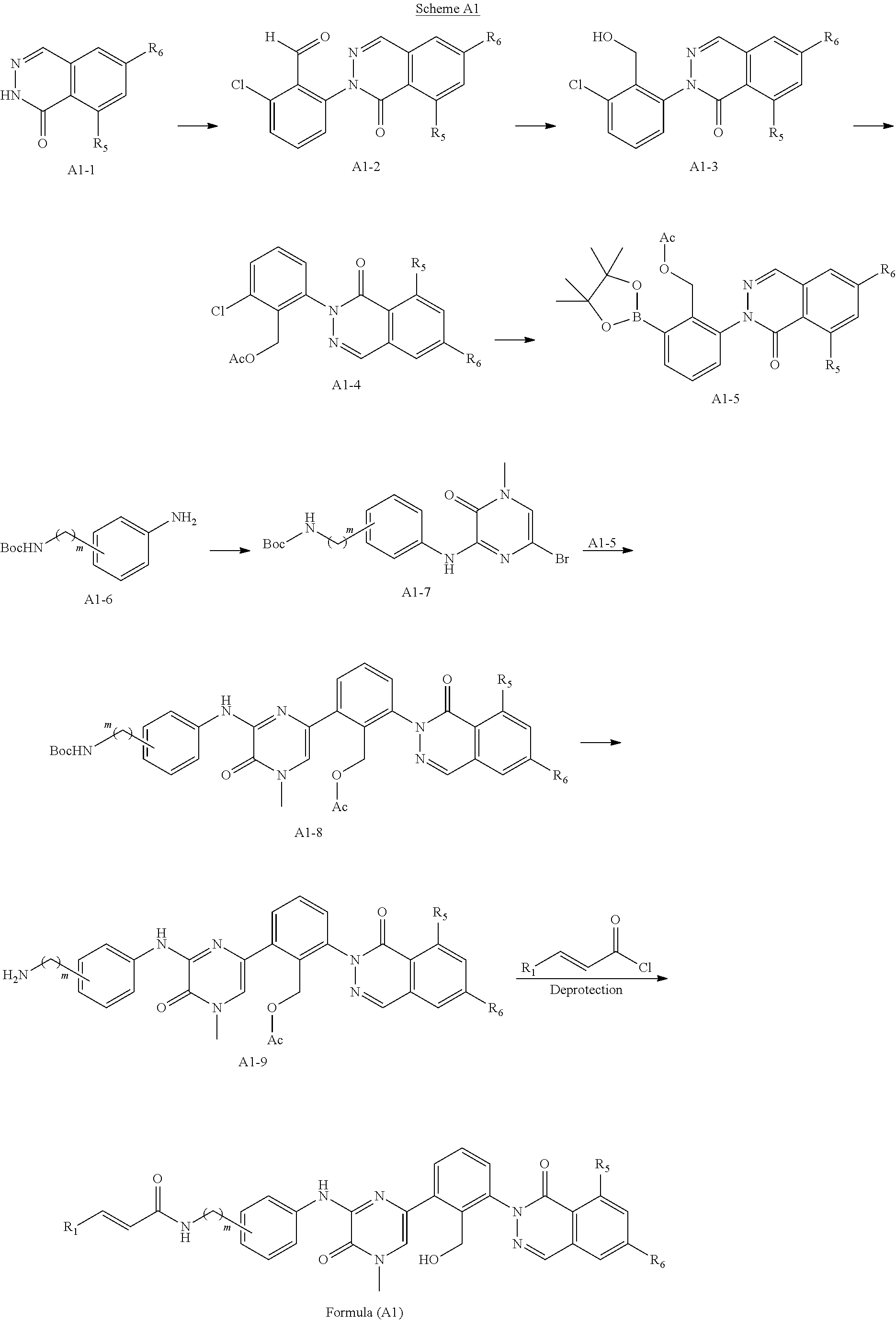

In Scheme A1, the starting material A1-1 can react with 2,6-halo-benzaldehyde to afford intermediate A1-2, which can be reduced to the alcohol intermediate A1-3. The protection of the hydroxyl group of A1-3 will lead to the intermediate A1-4, which can react with 4,4,4′,4′,5,5,5′,5′-octamethyl-2,2′-bi(1,3,2-dioxaborolane) to afford the intermediate A1-5. Meanwhile, the appropriate amine intermediate (A1-6) can react with 3,5-dibromo-1-methylpyrazin-2(1H)-one to yield the intermediate (A1-7), which can couple with A1-5 to form the intermediate A1-8. The de-protection of intermediate (A1-8) will affords the amine intermediate A1-9, which can react appropriate acryloyl chloride followed the deprotection of the Ac group to afford Formula (A1).

A typical approach to synthesize the Formula (C) compounds

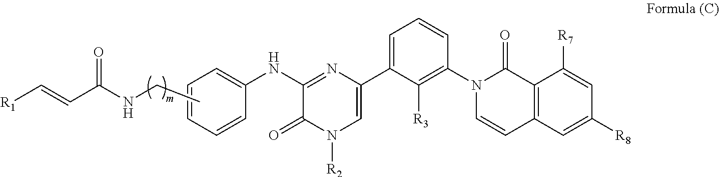

is described in Scheme C. R₁, R₂, R₃, R₇, R₈, and m in general Scheme C are the same as those described in the Summary section above.

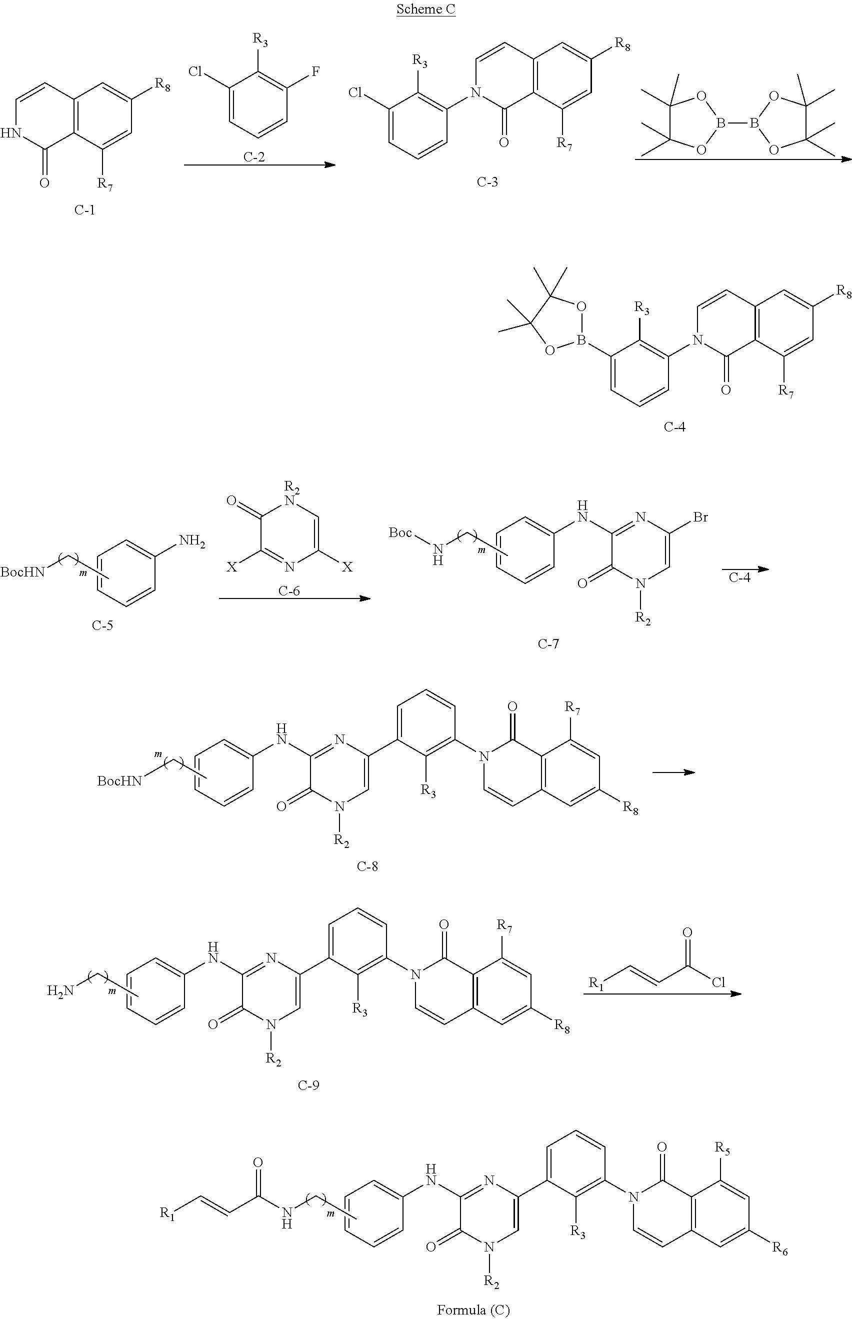

In Scheme C, the starting material C-1 can react with an appropriate 2-subsituted 1,3-halo-benzene C-2 to form the intermediate C-3, which can react with 4,4,4′,4′,5,5,5′,5′-octamethyl-2,2′-bi(1,3,2-dioxaborolane) to afford the intermediate C-4. Meanwhile, the appropriate amine intermediate (C-5) can react with 1-subsituted 3,5-dihalo-pyrazin-2(1H)-one (C-6), to yield the intermediate (C-7), which can couple with C-4 to form the intermediate C-8. Finally, the de-protection of intermediate (C-8) will affords the amine intermediate C-9, which can react appropriate acryloyl chloride to afford Formula (C).

Many kinds of starting material C-1 are commercially available. Alternatively, C-1 can be synthesized by various standard organic reactions, e.g.,

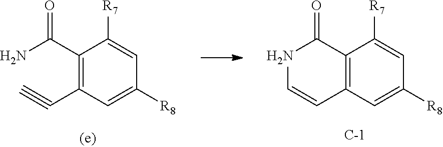

### (Varela-Fernandez, Alejandro et al., Synthesis, 44(21): 3285-3295 (2012)).

A typical approach to synthesize the Formula (E) compounds

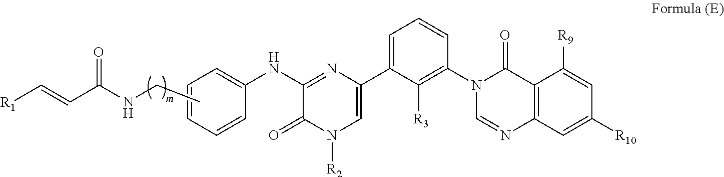

is described in Scheme E. R₁, R₂, R₃, R₉, R₁₀, and m in general Scheme E are the same as those described in the Summary section above.

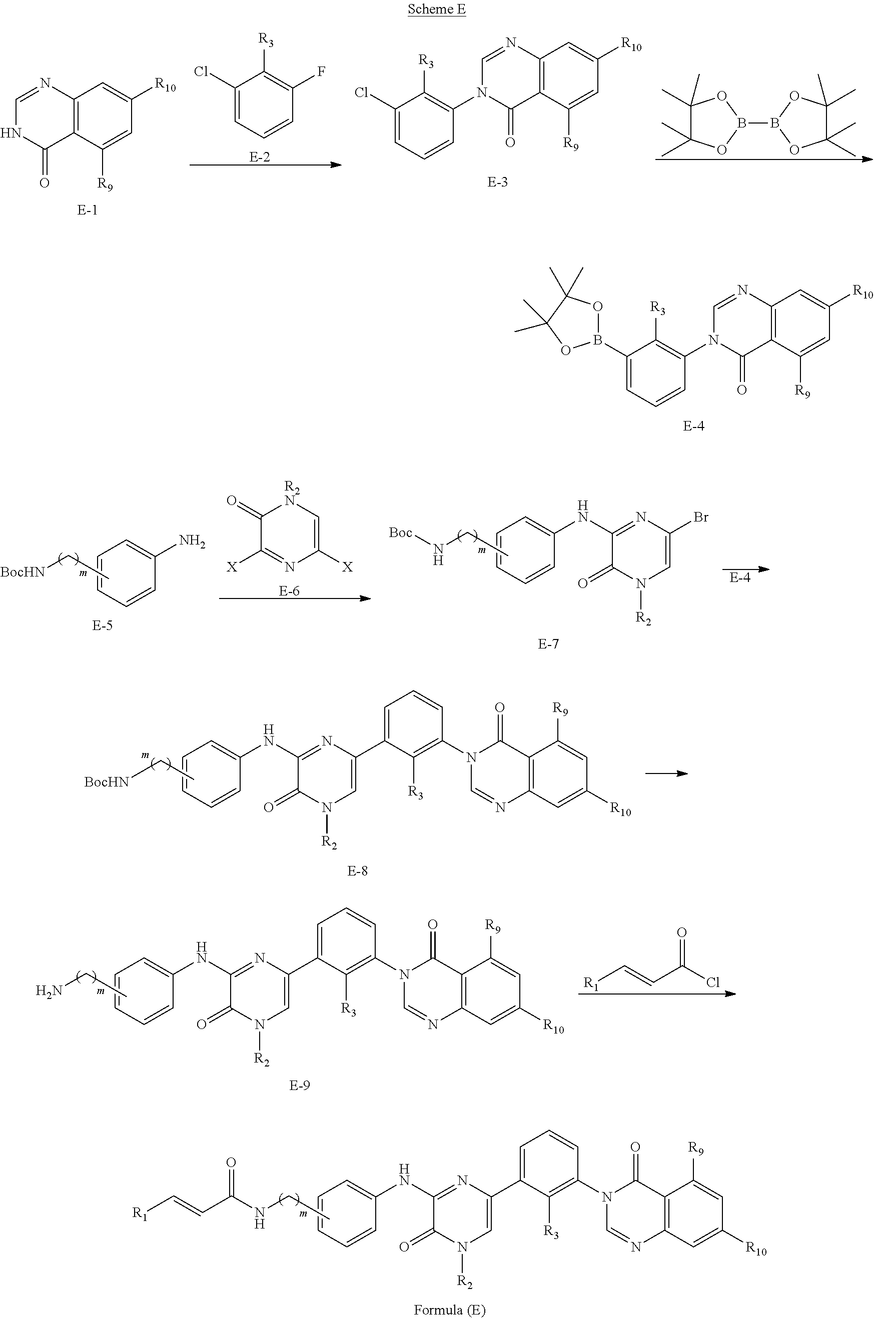

In Scheme E, the starting material E-1 can react with an appropriate 2-subsituted 1,3-halo-benzene E-2 to form the intermediate E-3, which can react with 4,4,4′,4′,5,5,5′,5′-octamethyl-2,2′-bi(1,3,2-dioxaborolane) to afford the intermediate E-4. Meanwhile, the appropriate amine intermediate (E-5) can react with 1-subsituted 3,5-dihalo-pyrazin-2(1H)-one (E-6), to yield the intermediate (E-7), which can couple with E-4 to form the intermediate E-8. Finally, the de-protection of intermediate (E-8) will affords the amine intermediate E-9, which can react appropriate acryloyl chloride to afford Formula (E).

Many kinds of starting material E-1 are commercially available. Alternatively, E-1 can be synthesized by various standard organic reactions, e.g.,

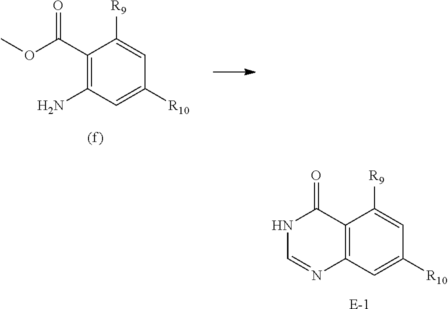

in which E-1 can be synthesized by reacting of appropriate 4,6 substituted 2-amino-benzoic acid with formamide.

A typical approach to synthesize the Formula (G) compounds

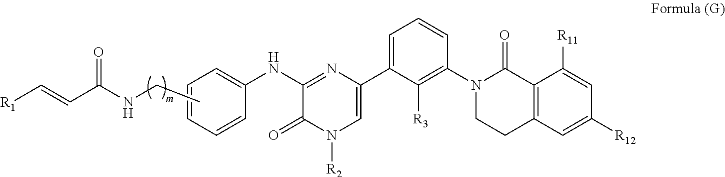

is described in Scheme G. R₁, R₂, R₃, R₁₁, R₁₂, and m in general Scheme G are the same as those described in the Summary section above.

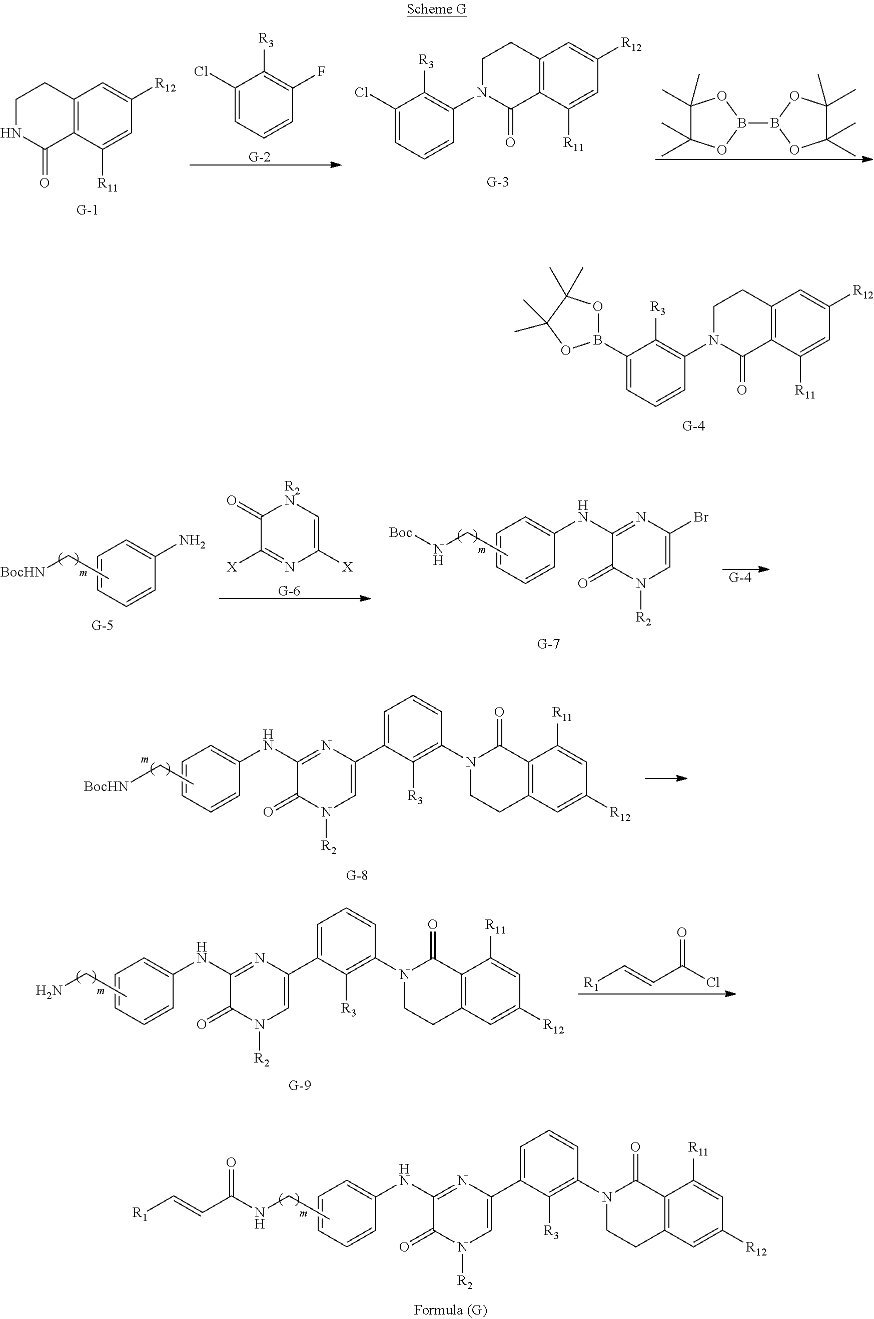

In Scheme G, the starting material G-1 can react with an appropriate 2-subsituted 1,3-halo-benzene G-2 to form the intermediate G-3, which can react with 4,4,4′,4′,5,5,5′,5′-octamethyl-2,2′-bi(1,3,2-dioxaborolane) to afford the intermediate G-4. Meanwhile, the appropriate amine intermediate (G-5) can react with 1-subsituted 3,5-dihalo-pyrazin-2(1H)-one (G-6), to yield the intermediate (G-7), which can couple with G-4 to form the intermediate G-8. Finally, the de-protection of intermediate (G-8) will affords the amine intermediate G-9, which can react appropriate acryloyl chloride to afford Formula (G).

Many kinds of starting material G-1 are commercially available. Alternatively, G-1 can be synthesized by various standard organic reactions, e.g.,

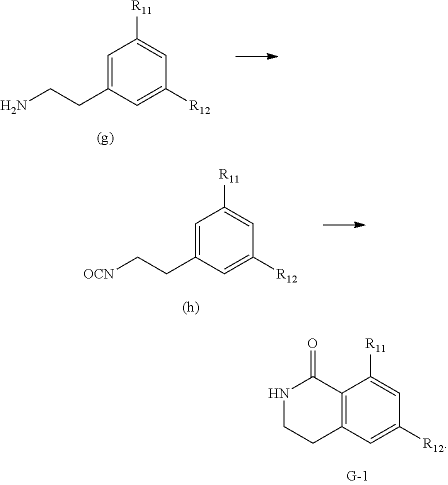

Treating compound of (g) with triphosgene will form a compound of (h), which react with a Lewis acid to undergo a cyclization reaction to form G-1.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example 1A

### Preparation of Key Intermediate 4

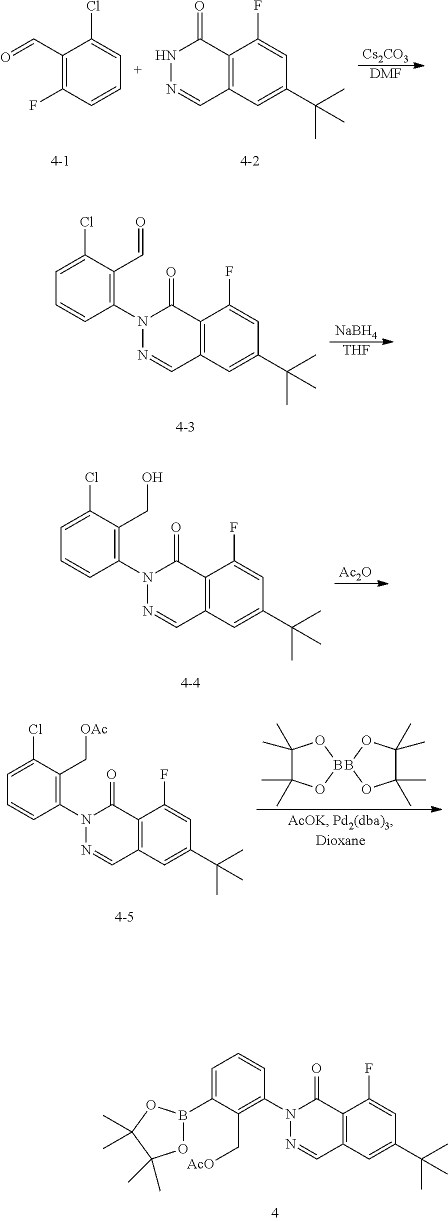

Procedure for Preparation of Compound 4-3:

To a 100 mL round bottom flask series added compound 4-1 (1.77 g, 8.04 mmol), commercially available starting material 4-2 (1.40 g, 8.84 mmol) and cesium carbonate (1.90 g, 4.82 mmol). The flask was evacuated and back filled with nitrogen three times. Then ethoxytrimethylsilane (1.90 g, 16.07 mmol) and DMF (20 mL) were added to the reaction flask, and the resulting mixture was heated 62° C. After 5 h stirring, the solution was allowed to cool down to ambient temperature and the reaction was quenched by addition 20 mL of water. The desired product started to precipitate from DMF and water mixture. The solid was collected by filtration after cooling down to 5° C., and washed with water. The filter cake was dried under vacuum oven at 50° C. to afford crude compound 3 (2.88 g, yield: 99.8%) as a light yellow solid which was used for next step without purification.

Procedure for Preparation of Compound 4-4:

To a solution of compound 4-3 (2.88 g, 8.03 mmol) in IPA/DCM (10/20 mL), then the resulting solution was cooled down to 4° C., and NaBH₄ (304 mg, 8.03 mmol) was added slowly. After 3 h stirring, the reaction mixture was quenched by added 20 mL of ice water and extracted with DCM (30 mL×3). The combined organic layers was washed with saturated brine and dried over Na₂SO₄, and concentrated under vacuum to give crude compound 4-4 which was purified by column chromatography to get compound 4-4 (1.84 g, yield: 64%) as a white solid.

Procedure for Preparation of Compound 4-5:

To a solution of compound 4-4 (1.84 g, 5.10 mmol) in 20 mL of DCM was added DMAP (125 mg, 1.02 mmol) at room temperature, then the resulting solution was cooled down to 4° C., and Ac₂O (1.04 g, 10.20 mmol) was added slowly. After 3 h stirring, TLC indicated compound 4-4 was completely consumed. The reaction mixture was in order washed with 1N HCl, saturated NaHCO₃ solution and saturated brine, dried over Na₂SO₄, then concentrated under vacuum to give compound 4-5 (2.05 g, yield: 100%) as a white solid.

Procedure for Preparation of Intermediate 4:

To a solution of compound 4-5 (1.4 g, 3.48 mmol) in 40 mL of dry dioxane was added 4,4,4′,4′,5,5,5′,5′-octamethyl-2,2′-bi(1,3,2-dioxaborolane) (2.65 g, 10.43 mmol), Sphos (856 mg, 2.09 mmol), AcOK (1.02 g, 10.43 mmol), Pd₂(dba)₃ (954 mg, 1.04 mmol) at room temperature. The flask was evacuated and back filled with nitrogen three times, and the resulting mixture was heated to 115° C. After 5 h stirring, the solution was allowed to cool down to ambient temperature and added 100 mL of EA, the organic phase was in order washed with saturated NaHCO₃ solution and saturated brine, dried over Na₂SO₄, then concentrated to give the crude product which was purified by preparative TLC to give compound 4 (850 mg, yield: 55%) as a white solid. ¹H NMR (CDCl3, 400 MHz): δ 8.17 (d, 1H), 7.96 (dd, 1H), 7.50-7.44 (m, 4H), 1.90 (s, 3H), 1.41 (s, 9H), 1.33 (s, 12H).

### Example 2A

### Preparation of CY-130526

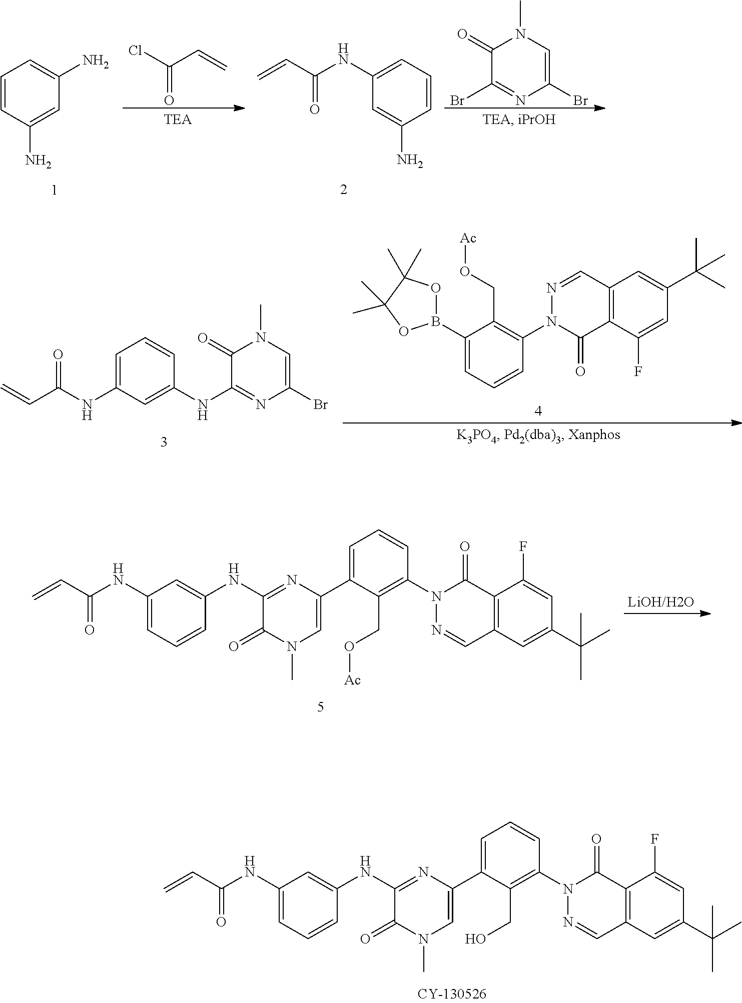

### Synthesis of Compound Cpd-2

To a solution of Cpd-1 (3.25 g, 30 mmol), Et₃N (3.3 g, 30 mmol) in DCM (150 mL) under nitrogen atmosphere was added acryloyl chloride (900 mg, 10 mmol) and the mixture was stirred at ambient temperature for 18 h. Saturated NaHCO3/water was carefully added (40 mL) and the mixture was stirred at ambient temperature for 1 h then extracted with dichloromethane. The aqueous phase was extracted with dichloromethane and the combined organic phase was washed brine, dried (Na₂SO₄), concentrated, dried and purified by Gel to provide Cpd-2 (0.8 g, 50%) as a yellow oil.

### Synthesis of Compound Cpd-3

To a solution of Cpd-2 (490 mg, 3 mmol) in iPrOH (20 ml) was added 3,5-dibromo-1-methylpyrazin-2(1H)-one (800 mg, 3 mmol). The reaction mixture was stirred at 90° C. for 24 h. The solvent was evaporated and purified by Gel to provide Cpd-3 (0.35 g, 30%) as a yellow solid.

### Synthesis of Compound Cpd-5

To a solution of Cpd-3 (70 mg, 0.2 mmol), Cpd-4 (100 mg, 0.2 mmol), xphos (20 mg, 0.05 mmol) and Pd₂(dba)₃ (20 mg, 0.05 mmol) in BuOH (10 mL) and water (2 mL) under nitrogen atmosphere was added K₃PO₄ (90 mg, 0.4 mmol) and the mixture was stirred at 115° C. for 5 h. The solvent was evaporated and purified by Gel to provide Cpd-5 (30 mg, 20%) as a white solid.

### Synthesis of Compound CY-130526

To a solution of Cpd-5 (30 mg) in THF (10 ml) and water (2 ml) was added LiOH (30 mg). The reaction mixture was stirred at rt for 2 h. The solvent was evaporated and purified by Gel to provide CY-130526 (20 mg, 75%) as a white solid. LC-MS(ESI): m/z=595[M+H]⁺ ¹H NMR (400 MHz, CDCl₃) δ 8.49 (s, 1H), 8.33 (s, 2H), 8.26 (d, J=2.4 Hz, 1H), 7.61 (d, J=6 Hz, 1H), 7.54 (m, 1H), 7.51 (m, 3H), 7.34 (d, J=8 Hz, 1H), 7.03 (m, 2H), 6.40-6.28 (m, 2H), 5.68 (dd, J=6, 1.6 Hz, 1H), 4.51 (d, J=6.4 Hz, 2H), 3.6 (s, 3H), 1.41 (s, 9H).

### Example 2B

### Preparation of CY-130589

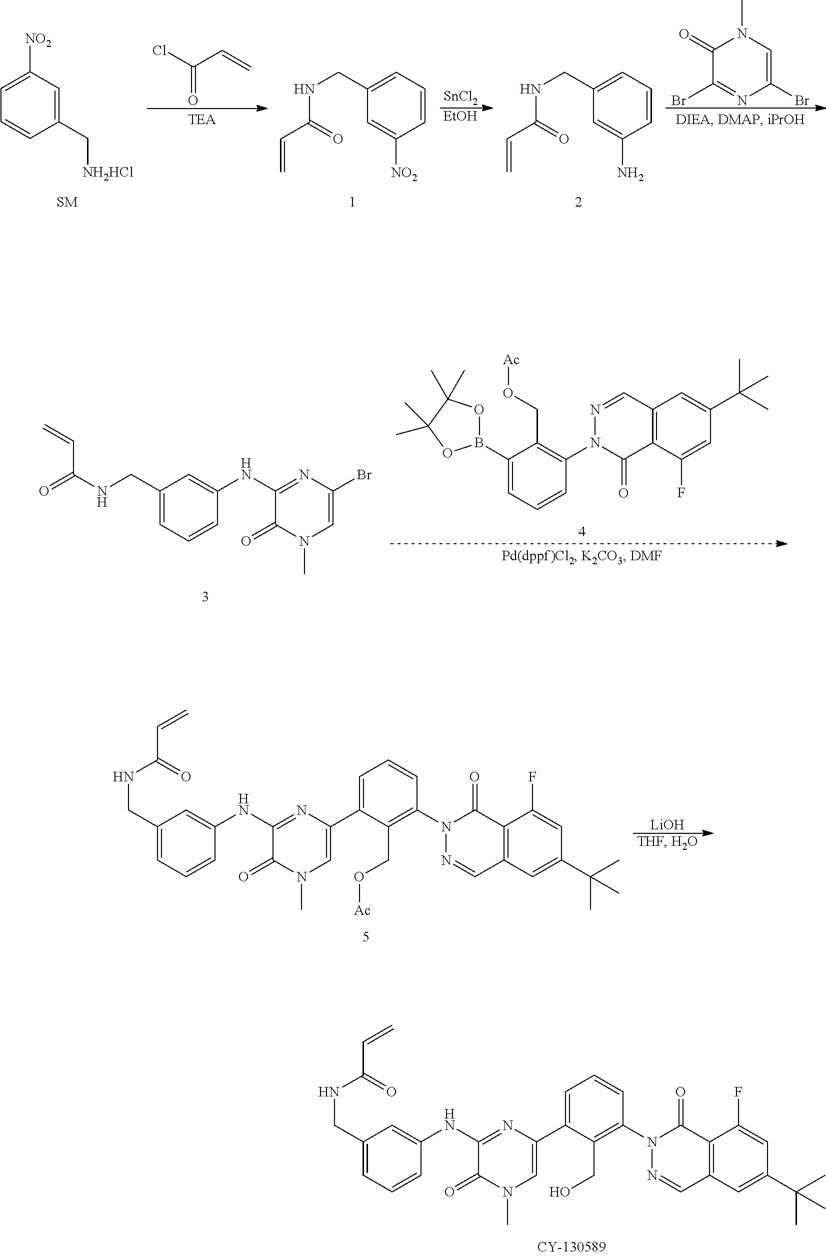

### Synthesis of N-(3-nitrobenzyl)acrylamide (1)

To a solution of SM (940 mg, 5 mmol) and TEA (505 mg, 5 mmol) in DCM (25 mL) was added drop wise acryloyl chloride (450 mg, 5 mmol) and the mixture was stirred at r.t overnight. The resulting solution was diluted with DCM (50 ml), washed with brine (50 ml×2), dried over anhydrous Na₂SO₄ and concentrated under vacuum to afford crude 1 (920 mg, 91%) as a light yellow solid. LC-MS (M+H)⁺=207.

### Synthesis of N-(3-aminobenzyl)acrylamide (2)

A solution of SnCl₂ (1.52 g, 8 mmol) in EtOH (50 mL) was stirred at 90° C. for 30 min and 1 (824 mg, 4 mmol) was added. The reaction solution was stirred at this temperature for 3 h. The resulting solution was concentrated and diluted with EA (200 ml), washed with 20% NaOH (300 ml), brine (250 ml), dried over anhydrous Na₂SO₄, concentrated under vacuum and purified by combi-Flash to afford 2 (390 mg, 55%) as a white solid. LC-MS (M+H)⁺=177.

### Synthesis of N-(3-(6-bromo-4-methyl-3-oxo-3,4-dihydropyrazin-2-ylamino)benzyl)acrylamide (3)

A solution of 2 (390 mg, 2.2 mmol), 3,5-dibromo-1-methylpyrazin-2(1H)-one (590 mg, 2.2 mmol), DIEA (303 mg, 3.3 mmol) and DMAP (27 mg, 0.22 mmol) in iPrOH (10 ml) was stirred at 80° C. overnight. The solvent was removed under reduced pressure and the residue was purified by combi-Flash to afford the product (500 mg, 64%) as a white solid. LC-MS (M+H)⁺=363.

### Synthesis of 2-(6-(3-(acrylamidomethyl)phenylamino)-4-methyl-5-oxo-4,5-dihydropyrazin-2-yl)-6-(6-tert-butyl-8-fluoro-1-oxophthalazin-2(1H)-yl)benzyl acetate (5)

To a solution of 3 (147 mg, 0.4 mmol), 4 (200 mg, 0.4 mmol) and K2CO3 (83 mg, 0.6 mmol) in DMF (10 ml) was bubbled N2 at room temperature for 30 min. Pd(dppf)Cl2 (20 mg) was then added and the reaction mixture was stirred at 90° C. under N2 overnight. The resulting suspension was filtered, concentrated under vacuum and purified by combi-Flash to afford 5 (140 mg, 55%) as a white solid. LC-MS (M+H)⁺=651.

### Synthesis of N-(3-(6-(3-(6-tert-butyl-8-fluoro-1-oxophthalazin-2(1H)-yl)-2-(hydroxymethyl)phenyl)-4-methyl-3-oxo-3,4-dihydropyrazin-2-ylamino)benzyl)acrylamide (CY-130589)

To a solution of 5 (140 mg, 0.22 mmol) in THF (5 mL) was added a solution of LiOH (18 mg, 0.44 mmol) in H2O (3 mL) and the reaction mixture was stirred at room temperature for 2 h. The resulting solution was adjusted pH to 7 with 0.1 N HCl, diluted with EA (50 ml), washed with brine (50 ml), dried over anhydrous Na₂SO₄, concentrated under vacuum and purified by Prep-HPLC to afford CY-130589 (9.2 mg, 7%) as a light yellow solid. LC-MS (M+H)+=609. 1H NMR (400 MHz, MeOD) δ 8.49 (d, 1H), 7.92 (s, 1H), 7.85 (s, 1H), 7.73-7.67 (m, 2H), 7.58 (dd, 7.3 Hz, 2H), 7.46 (d, 1H), 7.32-7.27 (m, 2H), 7.01 (d, 1H), 6.17-6.13 (m, 2H), 5.54 (dd, 1H), 4.57 (d, 2H), 4.46 (s, 2H), 3.66 (s, 3H), 1.47 (s, 9H).

### Example 2C

### The Following Compounds were Prepared by Methods Analogous to Those Disclosed in Scheme A-H

| Example | Structure | m/z(MH⁺) |
|---|---|---|
| 1. | 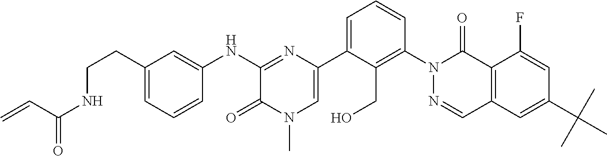 [SMILES: C=CC(=O)NCCc1cccc(Nc2nc(-c3cccc(-n4ncc5cc(C(C)(C)C)cc(F)c5c4=O)c3CO)cn(C)c2=O)c1] | 623 |
| 2. | 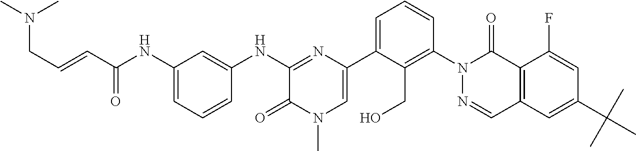 [SMILES: CN(C)C/C=C/C(=O)Nc1cccc(Nc2nc(-c3cccc(-n4ncc5cc(C(C)(C)C)cc(F)c5c4=O)c3CO)cn(C)c2=O)c1] | 652 |
| 3. | 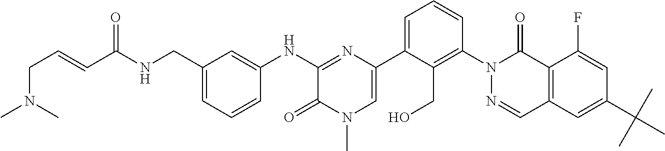 [SMILES: CN(C)C/C=C/C(=O)NCc1cccc(Nc2nc(-c3cccc(-n4ncc5cc(C(C)(C)C)cc(F)c5c4=O)c3CO)cn(C)c2=O)c1] | 666 |
| 4. | 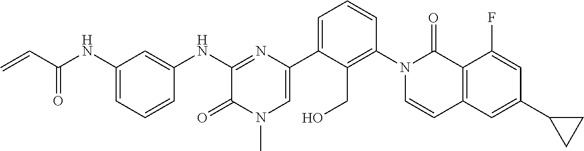 [SMILES: C=CC(=O)Nc1cccc(Nc2nc(-c3cccc(-n4ccc5cc(C6CC6)cc(F)c5c4=O)c3CO)cn(C)c2=O)c1] | 578 |
| 5. | 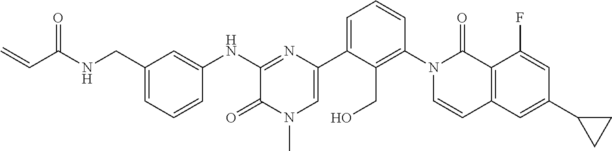 [SMILES: C=CC(=O)NCc1cccc(Nc2nc(-c3cccc(-n4ccc5cc(C6CC6)cc(F)c5c4=O)c3CO)cn(C)c2=O)c1] | 592 |
| 6. | 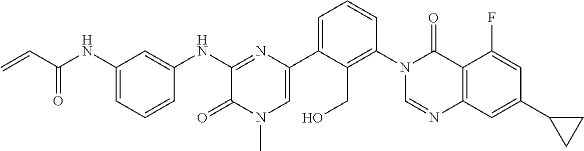 [SMILES: C=CC(=O)Nc1cccc(Nc2nc(-c3cccc(-n4cnc5cc(C6CC6)cc(F)c5c4=O)c3CO)cn(C)c2=O)c1] | 579 |
| 7. | 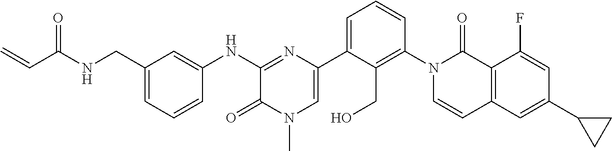 [SMILES: C=CC(=O)NCc1cccc(Nc2nc(-c3cccc(-n4ccc5cc(C6CC6)cc(F)c5c4=O)c3CO)cn(C)c2=O)c1] | 593 |
| 8. | 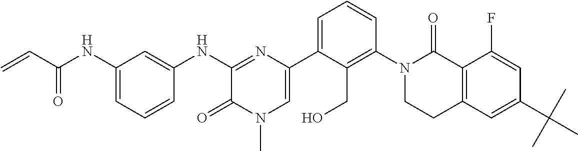 [SMILES: C=CC(=O)Nc1cccc(Nc2nc(-c3cccc(N4CCc5cc(C(C)(C)C)cc(F)c5C4=O)c3CO)cn(C)c2=O)c1] | 596 |
| 9. | 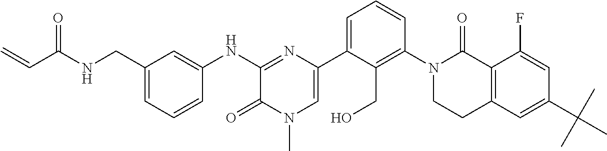 [SMILES: C=CC(=O)NCc1cccc(Nc2nc(-c3cccc(N4CCc5cc(C(C)(C)C)cc(F)c5C4=O)c3CO)cn(C)c2=O)c1] | 610 |

### Example 3

### Binding Constant (K_{d}) Determination and ScanMax Assay

The K_{d} of the compounds were determined by KINOMEscan™ assay, the industry's most comprehensive high-throughput system for screening compounds against large numbers of human kinases. KINOMEscan™ assay is based on a competition binding assay that quantitatively measures the ability of a compound to compete with an immobilized, active-site directed ligand. The assay is performed by combining three components: DNA-tagged kinase; immobilized ligand; and a test compound. The ability of the test compound to compete with the immobilized ligand is measured via quantitative PCR of the DNA tag. The kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR. An 11-point 3-fold serial dilution of each test compound was prepared in 100% DMSO at 100× final test concentration and subsequently diluted to 1× in the assay (final DMSO concentration=1%). Most K_{d} were determined using a compound top concentration=30,000 nM. If the initial Kd determined was <0.5 nM (the lowest concentration tested), the measurement was repeated with a serial dilution starting at a lower top concentration. A K_{d} value reported as 40,000 nM indicates that the K_{d} was determined to be >30,000 nM. Binding constants (K_{d}s) were calculated with a standard dose-response curve using the Hill equation: Response=Background+(Signal−Background)/[1+(K_{d}^{Hill Slope}/Dose^{Hill Slope})]. The Hill Slope was set to −1. Curves were fitted using a non-linear least square fit with the Levenberg-Marquardt algorithm. Such assays, carried out with a range of doses of test compounds, allow the determination of an approximate K_{d} value. Although the K_{d} of the compounds of the present invention vary with structural change as expected, the activity generally exhibited by these agents is in the range of K_{d}=0.1-10000 nM.

The following table lists the K_{d} values of CY-130526 and reference compound PCI-32765. The data clearly shows that CY-130526 is a highly potent BTK inhibitor. In addition, the kinases that were inhibited the most by CY-130526 next to BTK are TEC, BMX, with >1000-fold selectivity. These data suggests CY-130526 could have significantly less off-target toxicity than PCI-32765.

| | CY-130526 | PCI-32765 |
|---|---|---|
| Kd | (nM) | (nM) |
| BTK | 0.86 | 0.57 |
| BMX | 1,300 | 0.76 |
| JAK3 | 14,000 | 16.1 |
| TEC | 2,400 | 77.7 |

### Example 4

### Calcium Flux Fluoresence-Based Assay

Calcium flux fluorescence-based assays were performed in a FlexStation II384 fluorometric imaging plate reader (Molecular Devices) according to manufacturer instructions. In brief, actively growing Ramos cells (ATCC) in RPMI medium supplemented with 10% FBS (Invitrogen) were washed and re-plated in low serum medium at approximately 5×10⁵ cells per 100 μl per well in a 96-well plate. Compounds to be assayed were dissolved in DMSO and then diluted in low serum medium to final concentrations ranging from 0 to 10 μM (at a dilution factor of 0.3). The diluted compounds were then added to each well (final DMSO concentration was 0.01%) and incubated at 37 degree in 5% CO₂ incubator for one hour. Afterwards, 100 μl of a calcium-sensitive dye (from the Calcium 3 assay kit, Molecular Devices) was added to each well and incubated for an additional hour. The compound-treated cells were stimulated with a goat anti-human IgM antibody (80 ug/ml; Jackson ImmunoResearch) and read in the FlexStation II384 using a λ_{Ex}=485 nm and λ_{Em}=538 nm for 200 seconds. The relative fluorescence unit (RFU) and the IC₅₀ were recorded and analyzed using a built-in SoftMax program (Molecular devices).

### Example 5

### Inhibition of B-Cell Activation—B Cell FLIPR Assay in Ramos Cells

Inhibition of B-cell activation by compounds of the present invention is demonstrated by determining the effect of the test compounds on anti-IgM stimulated B cell responses. The B cell FLIPR assay is a cell based functional method of determining the effect of potential inhibitors of the intracellular calcium increase from stimulation by an anti-IgM antibody. Ramos cells (human Burkitt's lymphoma cell line. ATCC-No. CRL-1596) were cultivated in Growth Media (described below). One day prior to assay, Ramos cells were resuspended in fresh growth media (same as above) and set at a concentration of 0.5×10⁶/mL in tissue culture flasks. On day of assay, cells are counted and set at a concentration of 1×10⁶/mL1 in growth media supplemented with IμM FLUO-3AM (TefLabs Cat-No. 0116, prepared in anhydrous DMSO and 10% Pluronic acid) in a tissue culture flask, and incubated at 37° C. (5% CO₂) for one h. To remove extracellular dye, cells were collected by centrifugation (5 min, 1000 rpm), resuspended in FLIPR buffer (described below) at 1×10⁶ cells/mL and then dispensed into 96-well poly-D-lysine coated black/clear plates (BD Cat-No. 356692) at 1×10⁵ cells per well. Test compounds were added at various concentrations ranging from 100 μM to 0.03 μM (7 concentrations, details below), and allowed to incubate with cells for 30 min at RT. Ramos cell Ca²⁺ signaling was stimulated by the addition of 10 μg/mL anti-IgM (Southern Biotech, Cat-No. 2020-01) and measured on a FLIPR (Molecular Devices, captures images of 96 well plates using a CCD camera with an argon laser at 480 nM excitation).
Growth Medium: RPMI 1640 medium with L-glutamine (Invitrogen, Cat-No. 61870-010), 10% Fetal Bovine Serum (FBS, Summit Biotechnology Cat-No. FP-100-05); ImM Sodium Pyruvate (Invitrogen Cat. No. 11360-070).
FLIPR buffer: HBSS (Invitrogen, Cat-No. 141175-079), 2 mM CaCl₂ (Sigma Cat-No. C-4901), HEPES (Invitrogen, Cat-No. 15630-080), 2.5 mM Probenecid (Sigma, Cat-No. P-8761), 0.1% BSA (Sigma, Cat-No. A-7906), 11 mM Glucose (Sigma, Cat-No. G-7528);
Assay and Analysis: Intracellular increases in calcium were reported using a max-min statistic (subtracting the resting baseline from the peak caused by addition of the stimulatory antibody using a Molecular Devices FLIPR control and statistic exporting software. The IC₅₀ was determined using a nonlinear curve fit (GraphPad Prism).

### Example 6

### In Vitro Anti-Proliferation Assay

Cell antiproliferation is assayed by PerkinElmer ATPlite™ Luminescence Assay System. Briefly, the various test cancer cell lines are plated at a density of about 1×10⁴ cells per well in Costar 96-well plates, and are incubated with different concentrations of compounds for about 72 hours in medium supplemented with 5% FBS. One lyophilized substrate solution vial is then reconstituted by adding 5 mL of substrate buffer solution, and is agitated gently until the solution is homogeneous. About 50 μL of mammalian cell lysis solution is added to 100 μL of cell suspension per well of a microplate, and the plate is shaken for about five minutes in an orbital shaker at ˜700 rpm. This procedure is used to lyse the cells and to stabilize the ATP. Next, 50 μL substrate solution is added to the wells and microplate is shaken for five minutes in an orbital shaker at ˜700 rpm. Finally, the luminescence is measured by a PerkinElmer TopCount® Microplate Scintillation Counter. Such assays, carried out with a range of doses of test compounds, allow the determination of the cellular anti-antiproliferative IC₅₀ of the compounds of the present invention.

### Example 7

### In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

### Example 8

### Mouse Collagen-Induced Arthritis (mCIA)

On day 0 mice are injected at the base of the tail or several spots on the back with an emulsion of Type II Collagen (i.d.) in Complete Freund's adjuvant (CFA). Following collagen immunization, animals will develop arthritis at around 21 to 35 days. The onset of arthritis is synchronized (boosted) by systemic administration of collagen in Incomplete Freund's adjuvant (IFA; i.d.) at day 21. Animals are examined daily after day 20 for any onset of mild arthritis (score of 1 or 2; see score description below) which is the signal to boost. Following boost, mice are scored and dosed with candidate therapeutic agents for the prescribed time (typically 2-3 weeks) and dosing frequency, daily (QD) or twice-daily (BID). The developing inflammation of the paws and limb joints is quantified using a scoring system that involves the assessment of the 4 paws following the criteria described below:

Scoring:
1=swelling and/or redness of paw or one digit.
2=swelling in two or more joints.
3=gross swelling of the paw with more than two joints involved.
4=severe arthritis of the entire paw and digits.

Evaluations are made on day 0 for baseline measurement and starting again at the first signs or swelling for up to three times per week until the end of the experiment. The arthritic index for each mouse is obtained by adding the four scores of the individual paws, giving a maximum score of 16 per animal.

### Example 9

### Rat Collagen-Induced Arthritis (rCIA)

On day 0, rats are injected with an emulsion of Bovine Type II Collagen in Incomplete Freund's adjuvant (IFA) is injected intradermally (i.d.) on several locations on the back. A booster injection of collagen emulsion is given around day 7, (i.d.) at the base of the tail or alternative sites on the back. Arthritis is generally observed 12-14 days after the initial collagen injection. Animals may be evaluated for the development of arthritis as described below (Evaluation of arthritis) from day 14 onwards. Animals are dosed with candidate therapeutic agents in a preventive fashion starting at the time of secondary challenge and for the prescribed time (typically 2-3 weeks) and dosing frequency, daily (QD) or twice-daily (BID). The developing inflammation of the paws and limb joints is quantified using a scoring system that involves the assessment of the 4 paws following the criteria as described above. Evaluations are made on day 0 for baseline measurement and starting again at the first signs or swelling for up to three times per week until the end of the experiment. The arthritic index for each mouse is obtained by adding the four scores of the individual paws, giving a maximum score of 16 per animal.

### Example 10

### Rat In Vivo Asthma Model

Male Brown-Norway rats are sensitized i.p. with 100 μg of OA (ovalbumin) in 0.2 ml alum once every week for three weeks (day 0, 7, and 14). On day 21 (one week following last sensitization), the rats are dosed q.d. with either vehicle or compound formulation subcutaneously 0.5 hour before OA aerosol challenge (1% OA for 45 minutes) and terminated 4 or 24 hours after challenge. At time of sacrifice, serum and plasma are collected from all animals for serology and PK, respectively. A tracheal cannula is inserted and the lungs are lavaged 3× with PBS. The BAL fluid is analyzed for total leukocyte number and differential leukocyte counts. Total leukocyte number in an aliquot of the cells (20-100 μï) is determined by Coulter Counter. For differential leukocyte counts, 50-200 ï of the sample is centrifuged in a Cytospin and the slide stained with Diff-Quik. The proportions of monocytes, eosinophils, neutrophils and lymphocytes are counted under light microscopy using standard morphological criteria and expressed as a percentage. Representative inhibitors of Btk show decreased total leucocyte count in the BAL of OA sensitized and challenged rats as compared to control levels.

What is claimed is:

1. A compound of Formula (I) or an N-oxide thereof, or a pharmaceutically acceptable salt, solvate, polymorph or tautomer of said compound of formula (I) or N-oxide thereof: 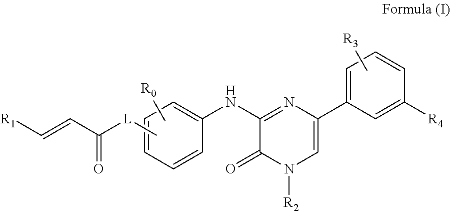
wherein
R₀ and R₁, independently, is H, alkyl, alkenyl, alkynyl, cycloalkyl, cycloalkenyl, heterocycloalkyl, heterocycloalkenyl, aryl, heteroaryl, halo, nitro, oxo, cyano, ORₐ, SRₐ, alkyl-Rₐ, alkyl-NR_{b}R_{c}, NR_{b}R_{c}, C(O)Rₐ, S(O)Rₐ, SO₂Rₐ, P(O)R_{b}R_{c}, C(O)N(R_{b})R_{c}, N(R_{b})C(O)R_{c}, C(O)ORₐ, OC(O)Rₐ, SO₂N(R_{b})R_{c}, or N(R_{b})SO₂R_{c}, in which each of Rₐ, R_{b}, and R_{c}, independently, is H, alkyl, alkenyl, alkynyl, cycloalkyl, cycloalkenyl, heterocycloalkyl, heterocycloalkenyl, aryl, heteroaryl, halo, cyano, amine, nitro, hydroxy, C(O)NHOH, alkoxy, alkoxyalkyl, haloalkyl, hydroxyalkyl, aminoalkyl, alkylcarbonyl, alkoxycarbonyl, alkylcarbonylamino, dialkylamino, or alkylamino;
L is —N(R_{d})(CH₂)ₘ in which R_{d} is H, alkyl, alkenyl, alkynyl, cycloalkyl, heterocycloalkyl; m, is 0, 1, 2, 3, or 4;
R₂ is H or alkyl;
R₃ is H, halo, alkyl, or hydroxyalkyl; and
R₄ is 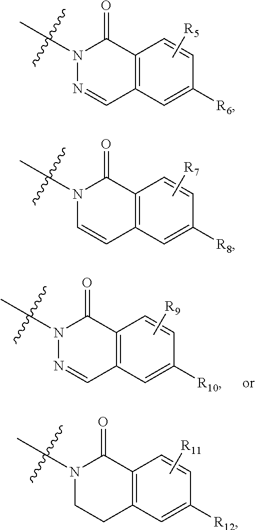
in which each of R₅, R₆, R₇, R₈, R₉, R₁₀, R₁₁, and R₁₂ independently, is H, alkyl, alkenyl, alkynyl, cycloalkyl, heterocycloalkyl, halo, or alkoxy.

2. A compound according to claim 1 or an N-oxide thereof, or a pharmaceutically acceptable salt, solvate, polymorph or tautomer thereof, wherein the compound is represented by

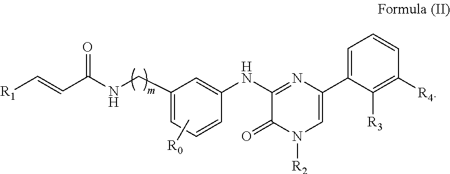

3. A compound according to claim 2 or an N-oxide thereof, or a pharmaceutically acceptable salt, solvate, polymorph or tautomer thereof, wherein R₄ is

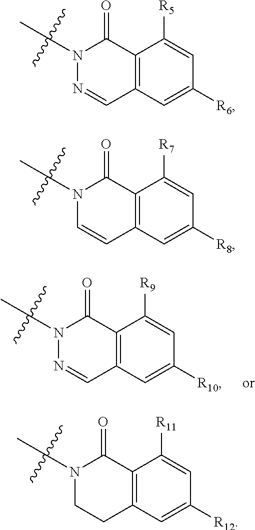

4. A compound according to claim 3 or an N-oxide thereof, or a pharmaceutically acceptable salt, solvate, polymorph or tautomer thereof, wherein R₁ is H, alkyl, alkyl-Rₐ, alkyl-NR_{b}R_{c}; R₂ is H, methyl, or ethyl; R₃ is H, methyl, or hydroxymethyl; and R₅, R₆, R₇, R₈, R₉, R₁₀, R₁₁, and R₁₂, independently, is H, alkyl, alkenyl, alkynyl, cycloalkyl, heterocycloalkyl, or halo.

5. A compound according to claim 4 or an N-oxide thereof, or a pharmaceutically acceptable salt, solvate, polymorph or tautomer thereof, wherein R₁ is H or alkyl-NR_{b}R_{c}; R₂ is methyl; R₃ is hydroxymethyl; R₆, R₈, R₁₀, and R₁₂, independently, is H, alkyl, or cycloalkyl; and R₅, R₇, R₉, and R₁₁ independently, is H, alkyl, or halo.

6. A compound according to claim 5 or an N-oxide thereof, or a pharmaceutically acceptable salt, solvate, polymorph or tautomer thereof, wherein R₅, R₇, R₉, and R₁₁ independently, is H or F.

7. A compound according to claim 1 or an N-oxide thereof, or a pharmaceutically acceptable salt, solvate, polymorph or tautomer thereof, wherein the compound is

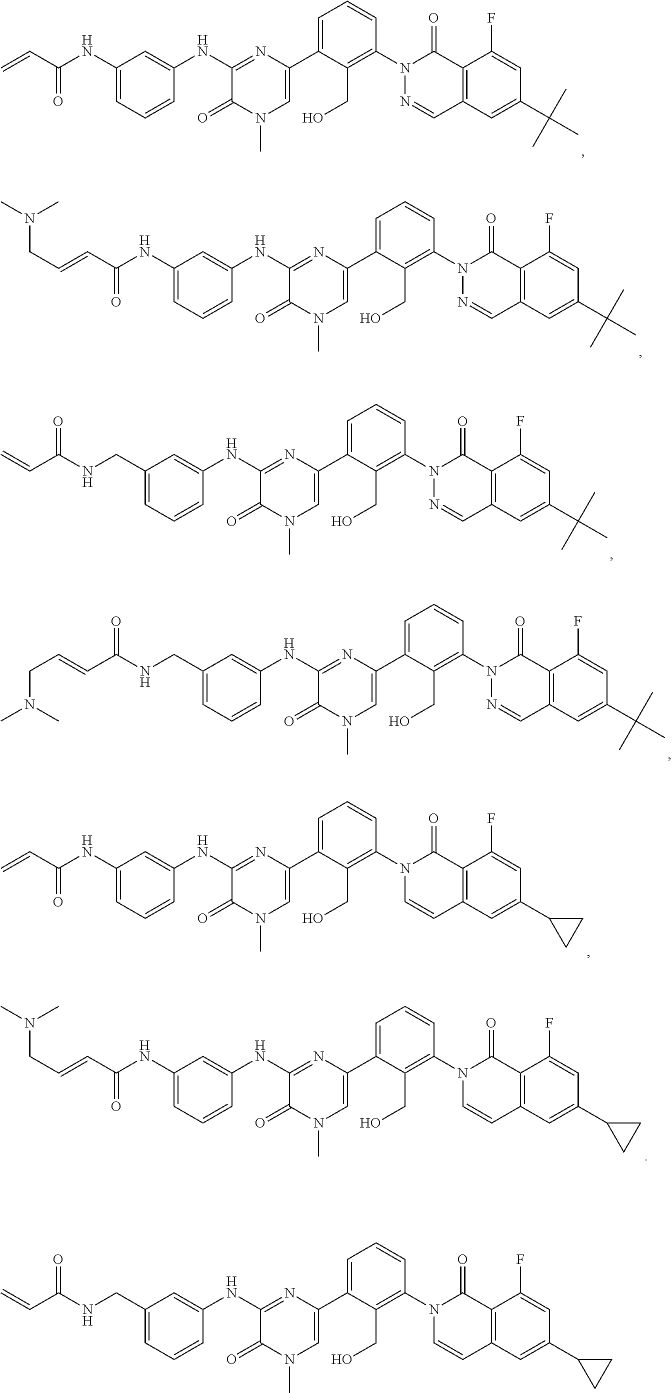

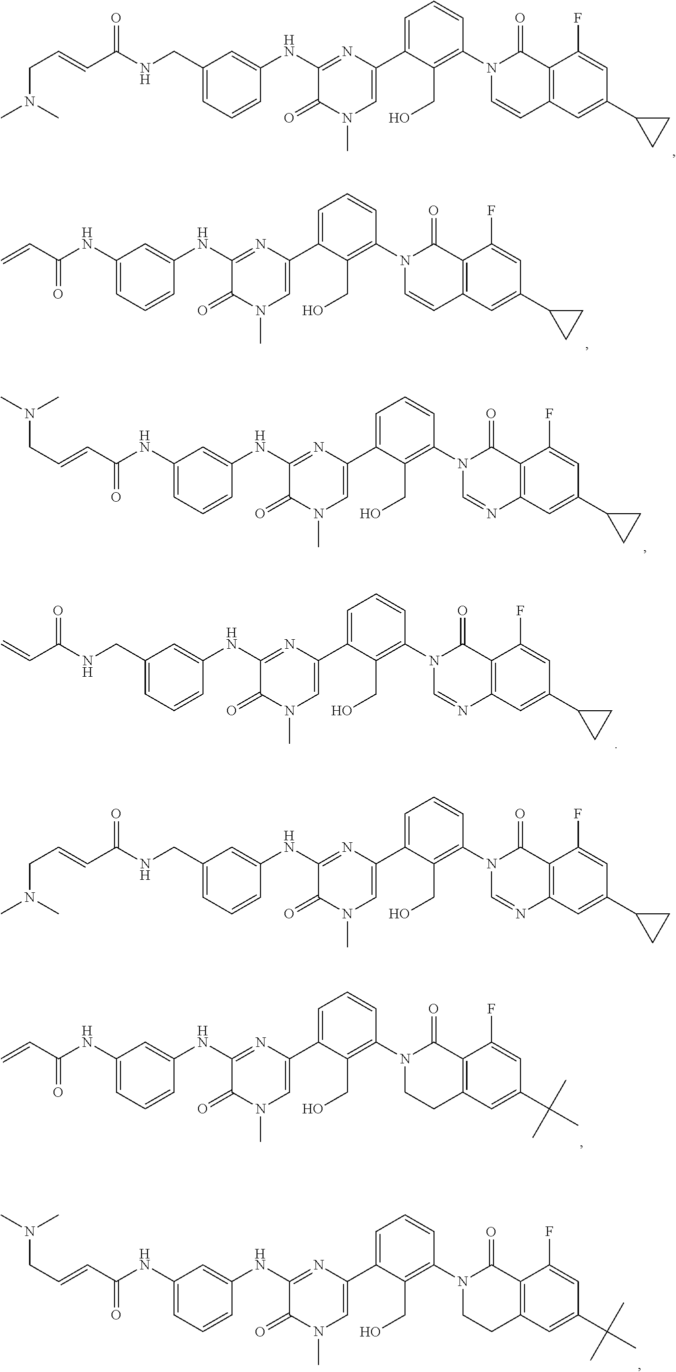

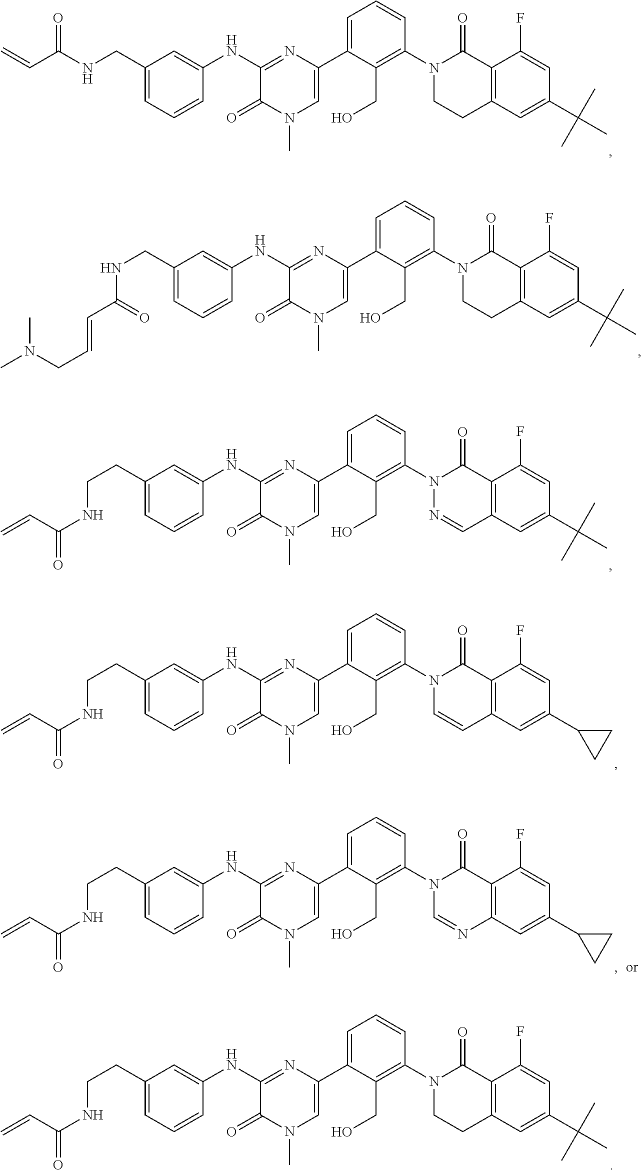

8. A compound according to claim 1 or an N-oxide thereof, or a pharmaceutically acceptable salt, solvate, polymorph or tautomer thereof, wherein the compound is

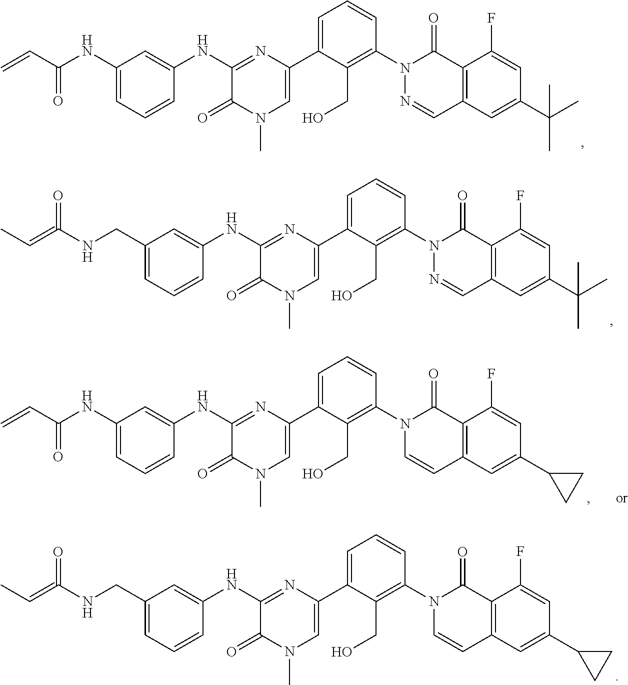

9. A compound according to claim 1 or an N-oxide thereof, or a pharmaceutically acceptable salt, solvate, polymorph or tautomer thereof, wherein the compound is

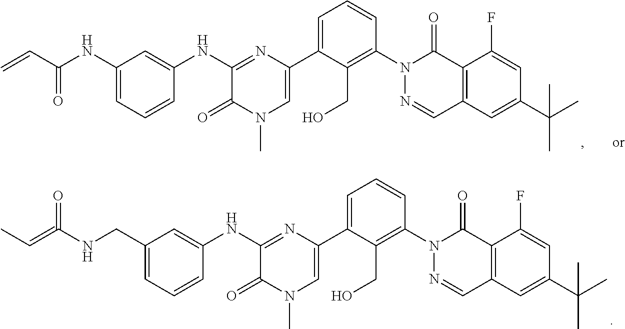

10. A pharmaceutical composition comprising a compound of formula (I) or an N-oxide thereof as defined in claim 1, or a pharmaceutically acceptable salt, solvate, polymorph or tautomer of said compound of formula (I) or an N-oxide thereof, and a pharmaceutically acceptable diluent or carrier.

11. A method of treating a neoplastic disease, autoimmune disease, and inflammatory disorder, comprising administering to a subject in need thereof an effective amount of a compound of formula (I) or an N-oxide thereof as defined in claim 1, or a pharmaceutically acceptable salt, solvate, polymorph or tautomer of said compound of formula (I) or an N-oxide thereof.

12. The method of claim 11, wherein said neoplastic disease, autoimmune disease, and inflammatory disorder is B-cell malignancy, rheumatoid arthritis (RA), asthma, multiple sclerosis, systemic lupus erythematosus, or allergy.

13. The method of claim 12, wherein said B-cell malignancy is chronic lymphocytic leukemia (CLL), small lymphocytic lymphoma (SLL), mantle cell lymphoma (MCL), and diffuse large B-cell lymphoma (DLBCL), or multiple myeloma (MM).
